(19) United States
(12) Patent Application Publication
(10) Pub. No.: US 2009/0318460 A1
(43) Pub. Date: Dec. 24, 2009

(54) AMORPHOUS VARENICLINE TARTRATE AND PROCESS FOR THE PREPARATION THEREOF

(75) Inventor: Mubeen Ahmed Khan, Navi Mumbai (IN)

Correspondence Address: GLENMARK PHARMACEUTICALS INC USA, 750 CORPORATE DRIVE, MAHWAH, NJ, 07430, US

(73) Assignee: GLENMARK, Mumbai (IN)

(21) Appl. No.: 12/468,883

(22) Filed: May 20, 2009

(30) Foreign Application Priority Data
May 26, 2008 (IN) 1106/MUM/2008
May 26, 2008 (IN) 1107/MUM/2008

(51) Int. Cl. A61K 31/4985; C07D 471/08

(52) U.S. Cl. 514/250; 544/343

(57) ABSTRACT

The present invention generally relates to an amorphous form of varenicline and its pharmaceutically acceptable salts thereof. More particularly the present invention relates to amorphous forms of varenicline and its L-tartrate salt; a process for their preparation and pharmaceutical compositions comprising the same.

### PRIORITY

This application claims the benefit to Indian Provisional Applications 1106/MUM/2008 (filed on May 26, 2008) and 1107/MUM/2008 (filed on May 26, 2008), the contents of each of which are incorporated by reference herein.

### BACKGROUND OF THE INVENTION

1. Technical Field

The present invention generally relates to amorphous forms of varenicline and its pharmaceutically acceptable salts thereof. More particularly the present invention relates to amorphous forms of varenicline and its L-tartrate salt; a process for their preparation and a pharmaceutical composition comprising the same.

2. Description of the Related Art

Varenicline tartrate is approved as an aid to smoking cessation treatment and is available in the market by brand name CHANTIX® in the form of tablet with the dosage strengths of 0.5 mg and 1.0 mg equivalent base. Varenicline tartrate is chemically known as 7,8,9,10-tetrahydro-6H-6,10-methano pyrazino[2,3-h][3]benzazepine L-tartrate and represented by structural formula I:

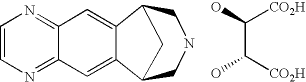

U.S. Pat. No. 6,410,550 describes aryl fused azapolycyclic compounds including varenicline.

U.S. Pat. No. 6,890,927 describes varenicline tartrate (L-; D-; D,L-; and meso), their polymorphs and processes for preparation thereof.

U.S. Pat. No. 7,265,119 describes crystalline anhydrous varenicline L-tartrate form A, anhydrous varenicline L-tartrate Form B, varenicline L-tartrate monohydrate form C.

U.S. Pat. No. 6,787,549 describes crystalline varenicline citrate hydrate form A, anhydrous form B and processes for preparation thereof

U.S. Pat. No. 6,794,388 describes varenicline succinate hydrate form and process for preparation thereof.

International application publication PCT WO 2008060487A2 describes crystalline forms A, C, D and E of Varenicline freebase.

### SUMMARY OF THE INVENTION

The present invention relates to amorphous forms of varenicline and its pharmaceutically acceptable salts thereof.

The present invention also relates to an amorphous form of varenicline L-tartrate.

The present invention also relates to a process for a preparation of amorphous forms of varenicline and its L-tartrate salt.

In an aspect, the present invention provides an amorphous form of varenicline characterized by an X-ray powder diffraction (XRPD) pattern that is substantially in accordance with FIG. 1.

In another aspect, the present invention provides amorphous forms of varenicline L-tartrate characterized by X-ray powder diffraction pattern that is substantially in accordance with FIG. 2.

In yet another aspect, the present invention provides a process for preparing amorphous forms of varenicline or a pharmaceutically acceptable salts thereof comprising:
(a) providing a solution of varenicline or a salt thereof in one or more solvents or aqueous mixtures; and
(b) removing the solvent(s) from the solution of (a); and
(c) recovering the varenicline or its L-tartrate salt substantially in amorphous form.

In yet another embodiment, the present invention relates to pharmaceutical compositions comprising amorphous varenicline or its pharmaceutically acceptable salts thereof and at least one pharmaceutically acceptable carrier.

### BRIEF DESCRIPTION OF THE ACCOMPANYING DRAWINGS

FIG. 1: shows a powder X-ray diffraction pattern for an amorphous form of varenicline.

FIG. 2: shows a powder X-ray diffraction pattern for an amorphous form of varenicline L-tartrate.

### DETAILED DESCRIPTION OF THE INVENTION

The present invention relates to amorphous form of varenicline.

The present invention also relates to amorphous form of varenicline L-tartrate.

The present invention also relates to a process for a preparation of amorphous varenicline and its L-tartrate salt

Polymorphism is the occurrence of different crystalline forms of a single compound and it is a property of some compounds and complexes. Thus, polymorphs are distinct solids sharing the same molecular formula, yet each polymorph may have distinct physical properties. Therefore, a single compound may give rise to a variety of polymorphic forms where each form has different and distinct physical properties, such as different solubility profiles, different melting point temperatures and/or different x-ray diffraction peaks. Since the solubility of each polymorph may vary, identifying the existence of pharmaceutical polymorphs is essential for providing pharmaceuticals with predicable solubility profiles. It is desirable to investigate all solid state forms of a drug, including all polymorphic forms, and to determine the stability, dissolution and flow properties of each polymorphic form. Polymorphic forms of a compound can be distinguished in a laboratory by X-ray diffraction spectroscopy and by other methods such as, infrared spectrometry.

The discovery of new amorphous forms of active pharmaceutical ingredients (APIs) provides opportunities to improve the performance characteristics of a pharmaceutical product. Such discoveries enlarge the repertoire of materials that a formulation scientist has available for designing, for example, a pharmaceutical dosage form of a drug with a targeted release profile or other desired characteristic properties.

Generally, amorphous solids offer opportunities for solubility and bioavailability enhancement since these materials are more soluble than the crystalline form of the same compound. The rate of dissolution is also a consideration in formulating syrups, elixirs and other liquid medicaments.

Additionally, polymorphic forms of the same drug substance or API, can be administered by itself or formulated as a drug product (also known as the final or finished dosage form), and are well known in the pharmaceutical art to affect, for example, the solubility, stability, flowability, tractability and compressibility of drug substances and the safety and efficacy of drug products.

Generally, amorphous materials do not exhibit the three dimensional long-range order ordinarily found in a crystalline material and are structurally more similar to liquids where the arrangement of molecules is random. Additionally, amorphous solids are not crystalline and therefore do not give a definitive x-ray diffraction pattern. Amorphous materials do not give rise to a melting point and tend to liquefy at some point beyond the glass transition point.

Neither an amorphous form of varenicline nor its L-tartrate salt has been heretofore described. The availability of either an amorphous form of varenicline or its L-tartrate salt would be beneficial in formulations geared towards the treatment of smoking cessation.

Thus, there is a need in the art, as yet heretofore unmet, for the discovery of amorphous forms of varenicline and its L-tartrate salt.

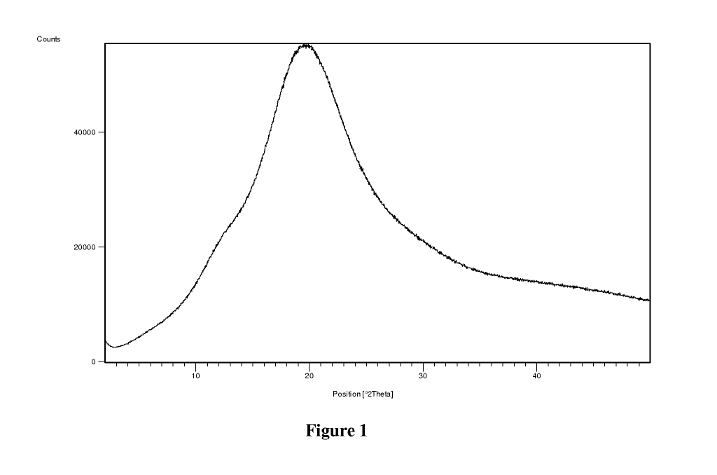

In an embodiment, the present invention provides amorphous form of varenicline characterized by X-ray powder diffraction pattern that is substantially in accordance with FIG. 1.

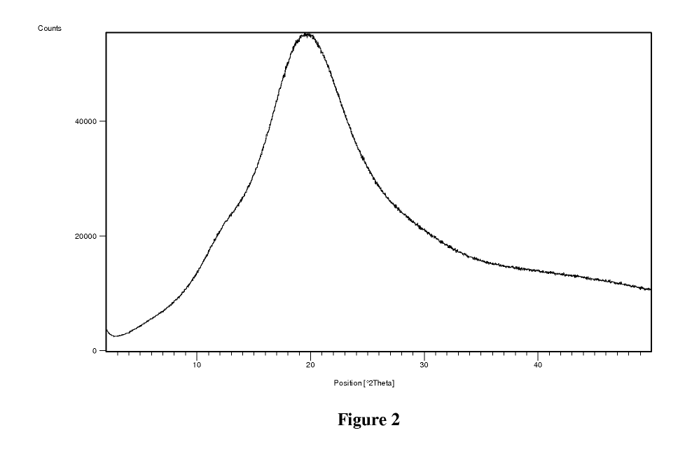

In another embodiment, the present invention provides an amorphous form of varenicline L-tartrate characterized by X-ray powder diffraction pattern that is substantially in accordance with FIG. 2.

In yet another embodiment, the present invention provides a process for preparing an amorphous form of varenicline or a pharmaceutically acceptable salt thereof comprising:
(a) providing a solution of varenicline or a salt thereof in one or more solvents or aqueous mixtures; and
(b) removing the solvent(s) from the solution; and
(c) recovering the varenicline or its L-tartrate salt substantially in amorphous form.

The solution of varenicline or its salt can be obtained by dissolving varenicline or a pharmaceutically acceptable salt thereof in a solvent or mixture of solvents or their aqueous mixtures thereof.

As used herein, a solvent is any liquid substance capable of dissolving varenicline or its salt.

As used herein a mixture of solvents refers to a composition comprising more than one solvent.

The solvents that can be used include, but are not limited to alcohols such as methanol, ethanol, n-propanol, isopropanol, n-butanol, isobutanol, and tertiary butyl alcohol and the like; ketonic solvents such as acetone, methyl ethyl ketone, methyl isobutyl ketone, 2-butanone and the like; halogenated solvents such as dichloromethane, ethylene dichloride, chloroform and the like; nitrile solvents such as acetonitrile, propionitrile and the like; esters such as ethyl acetate, isopropyl acetate and the like; aprotic polar solvents may include N,N-dimethylformamide (DMF), dimethylsulfoxide (DMSO), N,N-dimethyl acetamide (DMA) and the like; or mixtures thereof or their aqueous mixtures in various proportions without limitation. Preferably alcohols, halogenated solvents, ketones, nitriles and their aqueous mixtures. The volume of the solvent used to solubilize varenicline or a salt thereof may range from about 2 volumes to about 20 volumes to the weight of the varenicline or a pharmaceutically acceptable salt thereof taken. Preferably, from about 5 volumes to about 10 volumes.

The temperature for obtaining a clear and homogenous solution can range from about 25° C. to about 75° C. or the boiling point of the solvent/s used. Preferably, from about 25° C. to about 40° C.

The solution obtained is optionally filtered by using conventional filtration techniques known in the art, such as through celite or diatomaceous earth, to separate the extraneous matter present or formed in the solution.

Removal of solvent is accomplished by conventional techniques known in the art,for example, substantial evaporation of the solvent, concentrating the solution, cooling to obtain amorphous form and filtering the solid under inert atmosphere. Alternatively, the solvent may also be removed by evaporation. Evaporation can be achieved at sub-zero temperatures by the lyophilisation or freeze-drying technique. The solution may also be completely evaporated in, for example, a pilot plant rotavapor, a vacuum paddle dryer or in a conventional reactor under vacuum above about 720 mm Hg by flash evaporation techniques by using an agitated thin film dryer (ATFD), or evaporated by spray drying to obtain a dry amorphous powder. Preferably, the methods for drying are spray drying or vertical agitated thin-film drying (or evaporation).

In the preferred spray drying technique, a solution of varenicline or its pharmaceutically acceptable salt is sprayed into the spray drier at the flow rate ranging from about 10 ml/hr to about 300 ml/hr, preferably at flow rate of about 40 ml/hr to about 200 ml/hr. The air inlet temperature to the spray drier used may range from about 25° C. to about 150° C., preferably from about 60° C. to about 110° C. and the outlet air temperature used may range from about 30° C. to about 90° C. preferably from about 35° C. to about 50° C.

Illustratively, in a non-limiting example a typical spray drying apparatus comprises a drying chamber, atomizing means for atomizing a solvent-containing feed into the drying chamber, a source of drying gas that flows into the drying chamber to remove solvent from the atomized-solvent-containing feed, an outlet for the products of drying, and product collection means located downstream of the drying chamber. Examples of such apparatuses include Niro® Models PSD-1, PSD-2 and PSD-4 (Niro A/S, Soeborg, Denmark). Typically, the product collection means includes a cyclone connected to the drying apparatus. In the cyclone, the particles produced during spray drying are separated from the drying gas and evaporated solvent, allowing the particles to be collected. A filter may also be used to separate and collect the particles produced by spray drying. Spray-drying may be performed in a conventional manner in the processes of the present invention (see, e.g., Remington: The Science and Practice of Pharmacy, 19th ed., vol. II, pg. 1627, 2006 herein incorporated by reference). The drying gas used in the invention may be any suitable gas, although inert gases such as nitrogen, nitrogen-enriched air, and argon are preferred. Nitrogen gas is a particularly preferred drying gas for use in the process of the invention. The varenicline product produced by spray-drying may be recovered by techniques commonly used in the art, such as using a cyclone or a filter.

Agitated thin film evaporation technology, on the other hand, involves separating the volatile component using indirect heat transfer coupled with mechanical agitation of the flowing film under controlled condition. In vertical agitated thin-film drying (or evaporation) (ATFD-V), the starting solution is fed from the top into a cylindrical space between a centered rotary agitator and an outside heating jacket. The rotor rotation agitates the downside-flowing solution while the heating jacket heats it.

The recovery of either the amorphous varenicline or its amorphous L-tartrate salt, prepare in the process herein described, can be performed by any conventional method known in the art, such as filtration, decantation and centrifugation.

Preferably, recovery comprises filtering, washing, and drying the solid. Washing is usually done with the same solvent used in the reaction.

The above steps may be repeated to obtain the amorphous varenicline or its L-tartrate at a desired purity.

The varenicline or its L-tartrate salt substantially in an amorphous form obtained by the above processes may be further dried in, for example, vacuum tray dryer, rotocon vacuum dryer, vacuum paddle dryer or pilot plant rotavapor, to further lower residual solvents. When implemented, the preferred instrument is a vacuum tray dryer.

The temperature for drying can range from about 25° C. to about 75° C., preferably from about 25° C. to about 40° C. under vacuum.

The drying can be carried out for any desired time, time periods from about 1 to 20 hours frequently being sufficient.

The characterization of the amorphous form of varenicline and its L-tartrate salt by X-ray powder diffraction, previously described, were performed on a Philips X'pert PRO Diffractometer using Cu Kα radiation (Cu Kα1=1.54060 Å). The X-ray source is operated at 45 kV and 40 mA. Spectra are recorded at start angle from 2° to 50° 2θ, a step size 0.0167° with a time per step of 1000 seconds.

The varenicline or its L-tartrate salt used as starting material in the processes described herein above, may be of indefinite morphology, i.e. crystalline or amorphous or mixture thereof or may be crude varenicline or its tartrate salt resulting from synthetic processing step known in the art. Illustratively, U.S. Pat. Nos. 6,410,550 and 6,890,927 are incorporated herein in their entirety by reference.

The present invention provides an amorphous form of varenicline that is substantially free of crystalline polymorphs of varenicline, which may have less than 20%, more preferably less than 10%, even more preferably less than about 5%, and most preferably less than 1%, of any one of crystalline forms A, C, D, and E of varenicline. In one example, the XRPD pattern of the amorphous varenicline is substantially in accordance with FIG. 1.

The present invention provides an amorphous form of varenicline L-tartrate that is substantially free of polymorphs of varenicline, which may have less than 20%, more preferably less than 10%, even more preferably less than about 5%, and most preferably less than 1%, of any one of crystalline forms A, B and C of varenicline L-tartrate. In one example, the XRPD pattern of the amorphous varenicline L-tartrate is substantially in accordance with FIG. 2.

The present invention further provides accordingly, D₉₀ particle size of the unformulated amorphous varenicline or its L-tartrate salt obtained by the process of present invention is used as starting material in preparing a pharmaceutical composition generally is less than 400 microns preferably less than about 200 microns, more preferably less than 150 microns, still more preferably less than about 50 microns and still more preferably less than about 15 microns.

Any milling, grinding micronizing or other particle size reduction method known in the art can be used to bring the amorphous varenicline or its L-tartrate salt into any desired particle size range as set forth above.

In another embodiment, the present invention provides pharmaceutical compositions comprising varenicline or its L-tartrate substantially in an amorphous form, and at least one pharmaceutically acceptable carrier. The resulting mixture may be manufactured in the form of a unit-dose formulation (i.e., a physically discrete unit containing a specific amount of active ingredient) such as a tablet or capsule. The pharmaceutical compositions may be in the form of suspensions, solutions, elixirs, aerosols, or solid dosage forms.

Suitable carriers include but are not limited to fillers, binders, lubricants, inert diluents, surface active/dispersing agents, flavorings, antioxidants, bulking and granulating agents, adsorbents, preservatives, emulsifiers, suspending and wetting agents, glidants, disintegrants, buffers and pre-adjusting agents, and colorants. Examples of carriers include celluloses, modified celluloses, cyclodextrins, starches, oils, polyols, sugar alcohols and sugars, and others.

Other excipients contemplated by the present invention include binders, such as acacia gum, pregelatinized starch, sodium alginate, glucose and other binders used in wet and dry granulation and direct compression tableting processes; disintegrants such as sodium starch glycolate, crospovidone, low-substituted hydroxypropyl cellulose and others; lubricants like magnesium and calcium stearate and sodium stearyl fumarate; flavorings; sweeteners; preservatives; pharmaceutically acceptable dyes and glidants such as silicon dioxide.

The process of present invention is simple, efficient, cost effective, ecofriendly, robust, reproducible, commercially viable to produce the desired amorphous form of varenicline and its L-tartrate.

Having described the invention with reference to certain preferred embodiments, other embodiments will become apparent to one skilled in the art from consideration of the specification. The disclosures of the references referred to in this patent application are incorporated herein by reference. The invention is further defined by reference to the following examples describing in detail the process and compositions of the invention. It will be apparent to those skilled in the art that many modifications, both to materials and methods, may be practiced without departing from the scope of the invention.

### Examples

### Example 1

### Preparation of Varenicline Amorphous by Spray Drying

Dissolve Varenicline (5 g) in methanol (50 ml) and water (50 ml) and subject to spray drying using spray dryer to afford 9 g of the title compound.

### Example 2

### Preparation of Varenicline Amorphous by Lyophilization

Dissolve Varenicline (5 g) in water (100 ml) to give clear solution and subject to lyophilization using lyophilizer to afford 4 g of the title compound.

### Example 3

### Preparation of Amorphous Varenicline L-Tartrate by Spray Drying

Dissolve Varenicline (10 g) and L(+) tartaric acid (10 g) in acetone (50 ml) and water (50) and subject to spray drying using spray dryer to afford 10 g of the title compound.

### Example 4

### Preparation of Amorphous Varenicline L-Tartrate by Lyophilization

Dissolve Varenicline L(+) tartrate (5 g) in water (100 ml) to give clear solution and subject to lylophlization using lyophilser to afford 4 g of the title compound.

1) Amorphous form of varenicline or a pharmaceutically acceptable salt thereof.

2) The compound of claim 1, wherein the pharmaceutically acceptable salt of varenicline is varenicline L-tartrate.

3) The amorphous varenicline of claim 1, is characterized by XRPD which is substantially in accordance with FIG. 1.

4) The amorphous varenicline L-tartrate of claim 2, is characterized by XRPD which is substantially in accordance with FIG. 3.

5) (canceled)

6) (canceled)

7) The process of claim 14, wherein the solvent is selected from the group consisting of alcoholic solvents, halogenated solvents, ketones, nitrites, and aqueous mixtures thereof.

8) The process of claim 7, wherein the solvents used for preparation of solution of varenicline or a salt thereof is methanol or acetone or acetonitrile and/or their aqueous mixtures.

9) The process of claim 14, wherein the temperature is from about 25° C. to about reflux temperature of the solvent or mixture of solvents used.

10) The process of claim 8, wherein the solvent is removed by spray drying or lyophilization or evaporation.

11) A pharmaceutical composition comprising amorphous form of varenicline or its L-tartrate salt and at least a pharmaceutically acceptable carrier.

12) (canceled)

13) The amorphous varenicline and its L-tartrate salt, as in any of claims 1 or 2, are substantially free of other polymorphs.

14) A process for preparing amorphous form of varenicline or a pharmaceutically acceptable salt thereof, described as in any of claims 1-4 or 13, comprising:
(a) providing a solution of varenicline or a salt thereof in one or more solvents or aqueous mixtures; and
(b) removing the solvent(s) from the solution; and
(c) recovering the varenicline or its L-tartrate salt substantially in amorphous form.
